(19) United States
(12) Patent Application Publication
(10) Pub. No.: US 2002/0107521 A1
(43) Pub. Date: Aug. 8, 2002

(54) Bur abrading tool and method of use

(76) Inventors: Thomas D. Petersen, La Mesa, CA (US); Kenneth H. Holko, San Diego, CA (US)

Correspondence Address: John R. Ross, III, Ross Patent Law Office, P. O. Box 2138, Del Mar, CA, 92014, US

(21) Appl. No.: 09/732,113

(22) Filed: Dec. 7, 2000

(51) Int. Cl. A61B 17/56

(52) U.S. Cl. 606/85; 606/79

(57) ABSTRACT

A bur-abrading tool for use with a rotating or oscillating drive unit. The unit includes a base abrader bit unit comprising a bit rod and bit substrate shape having a surface of revolution. A very large number of particles, comprised of a plurality of shapes having sharp edges, are brazed to the substrate shape utilizing a brazing material which binds the particles to the substrate shape creating burs. The brazing material at least partially coats the particles. In preferred embodiments the particles are comprised of small cut pieces of wire. Tools having various surfaces of revolution and various cut wire shapes are described.

This invention relates to cutting and abrading tools and especially to such tools with bur particle cutting surfaces.

### BACKGROUND OF THE INVENTION

Bur abrading tools are well known. These tools may be reciprocating, rotary or oscillating. The burs of the cutting or abrading surface may be machined into the tool substrate or particles of a material different from the tool substrate (e.g., harder) may be applied to the tool substrate to create burs. A well-known technique for producing bur-abrading tools is to machine fluted groves to form a cutting surface. However, these burs tend to produce a kickback reaction when the tool first contacts the material being cut. Tool manufacturers have attempted to minimize kickback by increasing the rotational speed of the fluted bur tool. This represents a safety concern for the operator when the particles or debris is accelerated away form the tool. In the case of surgical procedures the increased speed of the tool may mean that precise cuts are more difficult to make. Also, bur particle fracture, imbedment, and cut misdirection are aggravated by high rotational speeds.

Applicant has developed a very successful technique for applying bur materials to abrading tool substrates and has used this technique to produce reciprocating bur abrading tools primarily for cutting bone in surgical procedures. Techniques for making and using these tools are described in the following U.S. Pat. Nos. 5,135,533; 5,358,547; 5,649,994; 5,707,276 all granted to Applicant or Applicant and another. These patents are hereby incorporated herein by reference. These patents describe a technique for using a brazing technique for applying bur particles to a bur abrading tool substrate. The braze material disclosed especially for these surgical applications is bio-compatible, wear resistant, corrosion resistant and lubricious. What is needed are rotating or oscillating bur-abrading tools suitable for use in surgical applications.

### SUMMARY OF THE INVENTION

The present invention provides a bur-abrading tool for use with a rotating or oscillating drive unit. The unit includes a base abrader bit unit comprising a bit rod and bit substrate shape having a surface of revolution. A very large number of particles, comprised of a plurality of shapes having sharp edges, are brazed to the substrate shape utilizing a brazing material which binds the particles to the substrate shape creating burs. The brazing material at least partially coats the particles. In preferred embodiments the particles are comprised of small cut pieces of wire. Tools having various surfaces of revolution and various cut wire shapes are described.

### BRIEF DESCRIPTION OF THE DRAWINGS

FIG. 1 shows several embodiments of the present invention.

FIG. 2 shows wire shapes and sizes.

FIGS. 3 and 4 are microscopic photographs of a preferred embodiment, magnified 18× and 70× respectively.

### DETAILED DESCRIPTION OF PREFERRED EMBODIMENTS

Preferred embodiments of the present invention may be described by reference to the drawings.

Cut Wire Particles

Applicants have developed an excellent technique for preparing abrading particles of any of a very large number of desired materials, shapes and sizes. This preferred technique utilizes metal wire, preferably stainless steel of any of a variety of cross sectional shapes such as round, oval, semi-circular, square, rectangular, triangular or any of several polygonal cross sections. To produce the abrading particles the wire is cut into small sections with lengths about the same as the major cross section dimension or up to about twice the major cross section dimension. A good choice is a length of 1 to 2 times the major cross section dimension. Applicants have discovered that the utilization of several different lengths is preferable to having all pieces the same length. Wires of various cross section dimensions can also be used. Particles can be cut normal to the wire axis but the preferred cutting angle is about 45 degrees to the wire axis. In addition to stainless steel the wire may be of any of several materials such as hardenable stainless steel, precipitation hardenable stainless steel, tool steel, cast alloys, nickel base hardenable alloys, cobalt base alloys, and hardenable forms of corrosion resistant steels, nickel or cobalt base alloys or titanium based materials. Drawings showing top and side views of such particles are shown in FIG. 2. In a preferred embodiment a mixture of particles are prepared consisting of the particles described in Table 1 .

TABLE 1

| | Wire | | | |
|---|---|---|---|---|
| Wire | Major | Particle | Cut Angle | Percent |
| Shape | Dimension | Length | (Degrees) | Composition |
| Oval | 0.4 mm | 0.4 to 0.8 mm | 45 | 20 |
| Rectangular | 0.4 mm | 0.4 to 0.8 mm | 45 | 30 |
| Semi-Circular | 0.4 mm | 0.4 to 0.8 mm | 45 | 40 |
| Round | 0.4 mm | 0.4 to 0.8 mm | 45 | 10 |

A machine was assembled by Applicants to cut the wire bur particles. It consisted of a stepping motor to advance the wire from a roll to a cutting device. The cutting device is similar to a small paper cutter but is driven up and down by a second motor with a cam and a return spring. The wire drive dwell time (no advance of the wire) is varied to control bur particle length. The cutter motor speed is also varied to control particle length. The angle of the cut could be varied from about 20 degrees to about 90 degrees by swiveling the cutter with respect to the wire axis.

Another preferred particle batch is prepared using rectangular wire with dimensions of 0.010 inch×0.015 inch, with one half of the particles cut at lengths of 0.020 inch and the other half cut at lengths of 0.010 inch, the cuts being made at 45 degrees. Also, approximately pyramid-shaped particles can be made using wire having a triangle cross-section and alternately cutting the wire at 60 degrees and 120 degrees to the wire axis to produce particles with five points. Alternatively, the triangular wire may be driven in a triangular slot with a flat side on top. The wire is sheared simultaneously at two angles each 30 degrees to a normal to the top flat side to produce the pyramid shaped particle having five points.

First Preferred Embodiment

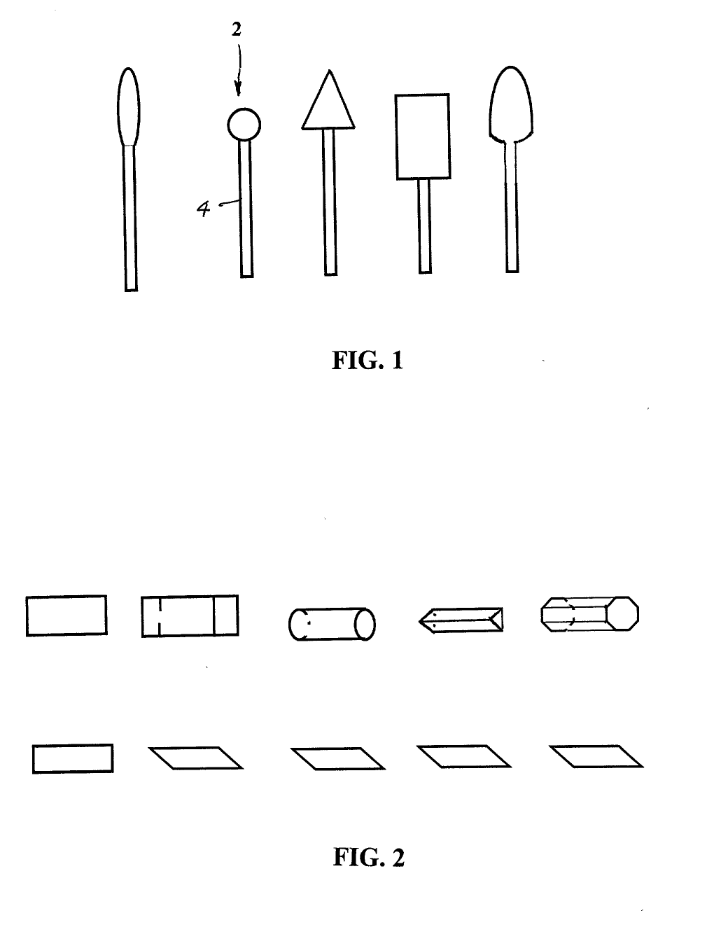

A first preferred embodiment of the present invention is a rotary abrading tool as shown at 2 in FIG. 1.

Fabrication Process

The preferred procedure for fabricating the tool is described below:

1) A base bit unit having the general shape shown at 2 in FIG. 1 comprising a ⅛ inch diameter bit rod 4 and a spherical bit abrader substrate having a radius of about ⅛ inch is machined from a 17-4PH stainless steel rod.

2) The spherical substrate is cleaned with acetone to remove any oil or other contaminants.

3) A binder comprised of a mixture of 75 percent acryloid 25 percent acetone is brushed on the substrate surface.

4) A minus 325 mesh mixture of 75 percent CoP and 25 percent Amdry 100 powders are dusted on the bur surface before the binder has dried.

5) A mixture of cut wire particles of age hardenable stainless steel as described in the preceding section are sprinkled on the coated surface.

6) Additional powder as described in Step 4 is dusted on top of the particles.

7) The coated base unit is put in a fixture to hold it with the axis of the tool in a vertical position.

8) The fixture with the tool is placed in a vacuum furnace and heated to 1975 degrees F. at a vacuum level of 1×10⁻⁴ torr or lower for 15 minutes.

9) The furnace is flooded with nitrogen and the tool is permitted to cool. The burs and abrasive particles are as the result of steps 3 through 9 now metallurgically bonded to the substrate surface and coated with the brazing material.

10) The tool is removed from the furnace and inspected. If additional cut wire particles are needed, steps 3 through 9 can be repeated.

11) If acceptable, the tool is next precipitation hardened. (Precipitation heat treatment is accomplished by heating the burs in a vacuum furnace to 925 degrees F. for 4 hours.)

12) The completed tools are then passivated, sterilized and packaged for shipment.

Completed Product Photographs

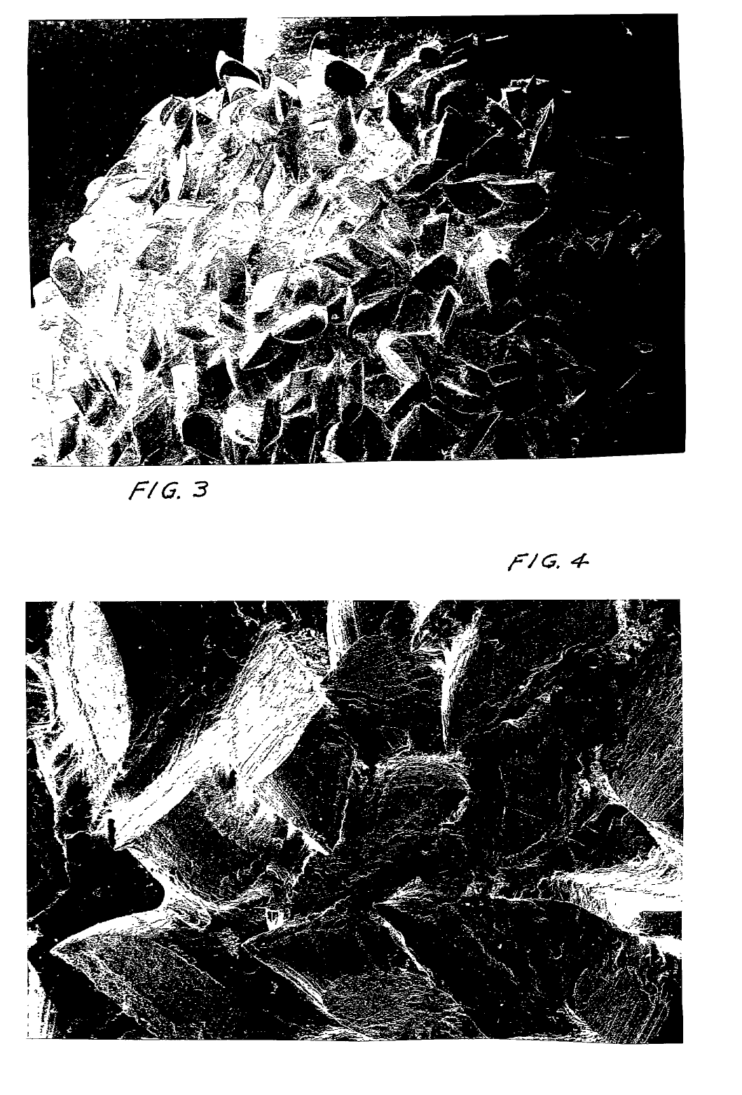

Microscopic photographs of a completed product produced in accordance with the above process is shown in FIGS. 3 and 4. FIG. 3 is magnified 18 times and FIG. 4 is magnified 70 times.

Applications

The completed tools shown in FIG. 1 have many applications. Some are designed specifically for bone shaping. One specific application is the removal of bone spurs near nerve roots. They may also be used for other medical applications such as dermabrasion and other soft tissue trimming and cutting. The multidirectional cutting action of these tools make them useful for dental and a variety of industrial applications.

Other Tool Shapes

The present invention covers many other tool shapes other than the spherical one discussed above. The tool can have any of the shapes shown in FIG. 1 or any other surface of revolution or partial surface of revolution. All of the tools however define an axis and in use they all move about the axis. The movement preferably is rotary, but it may also be oscillatory (i.e., back and forth) about the axis.

Other Cutting Particles

A preferred mixture of cutting particles is described above. However, many other sets of cutting particles could be substituted. For example, a single set of cut wire sections could be used such as 45 degree sections of the triangle cross section wire. Many wires other than stainless steel may be used depending on the application. As indicated above a variety of mixtures of shapes of cut particles can be used. Also, particles other than cut wire (such as diamonds, oxides, nitrides, aluminides and carbides including tungsten carbide) could be substituted. Some of these are discussed in U.S. Pat. No. 5,707,276. The size of the particles can be chosen to fit the particular application. Many wire sizes and shapes are available.

Other Brazing Materials

Many other brazing materials are available. These include the binders described in U.S. Pat. Nos. 5,707,276 and 5,358,547 such as Alloy No. 4: 75 to 90% CoP and the rest Nicrobraz 50; Alloy No. 7 which is 75% CoP and 25% Nicrobraz 135; Alloy No. 9 which is 75% CoP and 25% Amdry 100. Combinations of the above mixtures are also described in the '276 patent and may be substituted.

Uses of the Bur Abrading Tool

Applicant's tests have shown excellent results with the tools fabricated in accordance with the procedures set forth above.

A principal advantage of the present invention over prior art machined or fluted burs is the randomness of the particle edges. This avoids “kickback” or a recoil effect encountered when a fluted bur is first introduced to a hard surface for cutting. The lack of any significant kickback with burs of the present invention permits the tool to operate much more smoothly than the prior art devices and at lower speeds. This makes the cut more controllable and the procedure safer. When wire containing iron or other high permeability material is used, a magnet may be used to collect particles that may break off during the procedure. This can be a significant advantage in surgical procedures as compared to some prior art techniques.

Because of the lubricity of the coating, there is less tendency for the bur to fill or “load up” with cutting debris from the material being cut as compared with the prior art fluted bur devices.

The “aggressiveness” of the bur tool of the present invention can be easily varied by changing the bur particle size, shape or mixtures of sizes and shapes. This is typically difficult to do with conventional machined burs.

In addition to the embodiments described above the reader will recognize that there are many other possible variations to the preferred embodiments specifically described. Accordingly, the reader is requested to determine the scope of the invention by the appended claims and their legal equivalents, and not by the examples which have been given.

We claim:

1. A bur abrading tool for use with a rotating or oscillating drive unit, said tool comprising:
A) a base abrader bit unit comprising a bit rod and bit substrate shape, said rod and said substrate shape defining an axis,
B) a very large number of particles brazed to said substrate shape utilizing a brazing material which material binds said particles to said substrate shape and also coats at least partially said particles, said particles being comprised of a plurality of shapes having sharp edges and creating burs on said substrate shape.

2. A tool as in claim 1 wherein said particles are cut pieces of wire.

3. A tool as in claim 2 wherein said wire is stainless steel wire.

4. A tool as in claim 2 wherein said wire is cut at angles of about 45 degrees.

5. A tool as in claim 2 wherein said wire defining cross sections and said cross sections include at least one of the cross sections in a group of cross sections consisting of: square, triangular, circular, semi-circular and rectangular and define a major dimension.

6. A tool as in claim 5 wherein said cross sections include at least two of the cross sections in a group of cross sections consisting of: square, triangular, circular, semi-circular and rectangular and define a major dimension.

7. A tool as in claim 5 wherein said cross sections include at least three of the cross sections in a group of cross sections consisting of: square, triangular, circular, semi-circular and rectangular and define a major dimension.

8. A tool as in claim 2 wherein most of said particles have a length and a major cross section dimension and said length is longer than one half said major dimension but shorter than twice said major dimension.

9. A tool as in claim 2 wherein said particles have a variety of lengths.

10. A tool as in claim 9 wherein most of said particles have a variety of lengths, wherein most of said variety of lengths are longer than one half said major dimension but shorter than twice said major dimension.

11. A tool as in claim 1 wherein about half of said particles have a length of X and about half of said particles have a length of 2X.

12. A tool as in claim 1 wherein said particles are fully covered with said brazing material.

13. A tool as in claim 1 wherein said wire is comprised of a material chosen from a group consisting of: hardenable stainless steel, precipitation hardenable stainless steel, tool steel, cast alloys, nickel base hardenable alloys, cobalt base alloys, and hardenable forms of corrosion resistant steels or nickel or cobalt base alloys, titanium based materials.

14. A tool as in claim 1 wherein said brazing material is chosen from a group consisting of: Alloy No. 4, Alloy No. 7, Alloy No. 9, 60% alloy no. 4 plus 40% Amdry 100; 60% Alloy No. 7 plus 40% Amdry 100 and 55% Nicrobraz 50 plus 45% Nicrobraz 135.

15. A process for abrading a material comprising the steps of:
A) attaching to a rotary drive unit a bur abrading tool, said tool comprising:
1) a base abrader bit unit comprising a bit rod and bit substrate shape, said rod and said substrate shape defining an axis and
2) a very large number of particles brazed to said substrate shape utilizing a brazing material which material binds said particles to said substrate shape and also coats at least partially said particles, said particles being comprised of a plurality of shapes having sharp edges;
B) using said drive unit and said tool to abrade a material.

16. A process as in claim 15 wherein said material is bone.

17. A process for abrading a material comprising the steps of:
A) attaching to an oscillating drive unit a bur abrading tool, said tool comprising:
1) a base abrader bit unit comprising a bit rod and bit substrate shape, said rod and said substrate shape defining an axis and
2) a very large number of particles brazed to said substrate shape utilizing a brazing material which material binds said particles to said substrate shape and also coats at least partially said particles, said particles being comprised of a plurality of shapes having sharp edges;
C) using said drive unit and said tool to abrade a material.

18. A process as in claim 17 wherein said material is bone.
